(19) United States
(12) Patent Application Publication
(10) Pub. No.: US 2026/0033883 A1
(43) Pub. Date: Feb. 5, 2026

(54) DEVICE AND METHOD TO TREAT CHRONIC LOWER BACK PAIN VIA SINUVERTEBRAL NERVE ABLATION

(71) Applicant: AVENT, INC., Alpharetta, GA (US)

(72) Inventors: Eric A. SCHEPIS, Alpharetta, GA (US); Natalia ALEXEEVA, Alpharetta, GA (US); Lee C. BURNES, Alpharetta, GA (US)

(21) Appl. No.: 18/998,314

(22) PCT Filed: Jul. 26, 2023
(86) PCT No.: PCT/US2023/028678

§ 371 Date: Jan. 24, 2025

Related U.S. Application Data
(60) Provisional application No. 63/392,576, filed on Jul. 27, 2022.

(51) Int. Cl. A61B 18/12; A61B 18/00

(52) U.S. Cl. CPC A61B 18/12; A61B 2018/00029; A61B 2018/00434; A61B 2018/00577; A61B 2018/00648; A61B 2018/00964

(57) ABSTRACT

A probe for radiofrequency (RF) tissue ablation includes an elongated shaft that extends from a proximal end to a distal end, the elongated shaft comprising a bent portion at the distal end, and a tip positioned on the distal end of the elongated shaft after the bent portion, the tip comprising an active side and an inactive side, the inactive side opposite the active side and comprising an insulating material, the active side comprising one or more electrodes extending therefrom for delivering RF energy to a target nerve. The probe can be connected to an RF generator for producing RF energy to be de-livered via the active side of the tip.

### CROSS-REFERENCE TO RELATED APPLICATIONS

This application claims priority to and the benefit of U.S. Provisional Patent App. No. 63/392,576, filed Jul. 27, 2022, which is incorporated herein by reference in its entirety.

### BACKGROUND

Chronic lower back pain (CLBP) has a multicausal pathology and is thought to originate in the intervertebral discs, vertebral bodies, facet joints, and myofascial ensemble. Conservative approaches to CLBP management include NSAIDs, physical therapy, local injections of anesthetics and steroids, opioids, electrical stimulation, and nerve ablation. Surgical treatment options (i.e., disc fusions, replacements, etc.) exist and are time-consuming, expensive, risky, and often ineffective at treating pain. Nerve ablation techniques to treat CLBP, which may circumvent some disadvantages of surgical treatments, can include: medial nerve branch denervation of the zygapophysial (facet) joint (e.g., to treat pain from the joint); basivertebral nerve ablation to treat vertebrogenic pain (i.e., pain from the vertebral bodies); and intradiscal electrothermal annuloplasty and disc biacuplasty to treat discogenic pain (i.e., pain from the discs).

Medial nerve branch denervation procedures are routinely performed across the globe. The procedure is minimally invasive and only affects soft tissues (e.g., nerves, fascia, muscle, fat, skin, etc.). Additionally, it is known to be a low-risk procedure that is performed under fluoroscopic or ultrasound guidance, and with low surgical burden. The treatments for vertebrogenic and discogenic pain are considerably more invasive and time-consuming compared to the medial nerve branch denervation procedure and are risker to both the patient and health care provider. That is, they require that the ablative probes be passed through diseased vertebral bone or intervertebral disc to access the target nerve.

The basivertebral nerve is formed at center-midline on the posterior border of the vertebral body and is assessed through the bony pedicle (transpedicular intraosseous approach). Likewise, the physician must bore a hole through the pedicle of the diseased vertebral body to access the targeted basivertebral nerve at each bony level that will be treated. Since CLBP has a multilevel pathology, the provider must bore holes at multiple bony levels to effectively manage pain. Studies investigating transpedicular intraosseous approach to basivertebral nerve ablation for CLBP management have been conducted and demonstrate a 60% decrease in Oswestry Disability Index (ODI) score at 3-month, 6-month, and 1-year post-treatment, with some effects lasting for 5-years. Despite the prolonged effects, the magnitude of pain relief was only marginal, suggesting that the ablation only partially treated the basivertebral nerve branch.

Intradiscal electrothermal annuloplasty (IDET) and disc biacuplasty treats CLBP by destroying the nerve fibers located in the posterior third of a vertebral disc. IDET is performed by advancing a curved probe into the diseased intervertebral disc where it then wraps around the annulus fibrosis. By heating the probe, the nociceptive fibers of the IVD are destroyed, and the collagen of the disc is modified to prevent further damage. Alternatively, in disc biacuplasty, two needle-like probes are inserted into the posterior intervertebral disc and used to thermally ablate nerve fibers that are located in the posterior disc.

These two procedures showed minor success on a long-term scale, with IDET resulting in a 5-point ODI decrease at 17.1 months post-treatment, and disc biacuplasty having a 6.8-point ODI decrease at 1-month post-treatment. This ODI drop remained similar through 6-months post-treatment. Recovery from these procedures is substantial compared to the less invasive ablation procedures: IDET has a four-month recovery window, while disc biacuplasty recovery is on average 12-weeks. Much like the basivertebral nerve ablation, these approaches to CLBP must be repeated at each level, resulting in a long procedure time. Overall, the discogenic treatment options are time and risk intensive, have a long recovery window, and fail to comprehensively treat CLBP.

In summary, the techniques described above suffer various disadvantages including the further destruction of diseased bone and tissue, lengthy surgical times, intraoperative risks, and marginal treatment efficacy.

### SUMMARY

One implementation of the present disclosure is a probe for radiofrequency (RF) tissue ablation, the probe including: an elongated shaft that extends from a proximal end to a distal end, the elongated shaft including a bent portion at the distal end; and a tip positioned on the distal end of the elongated shaft after the bent portion, the tip including an active side and an inactive side, the inactive side opposite the active side and including an insulating material, the active side including one or more electrodes extending therefrom for delivering RF energy to a target nerve.

In some implementations, the bent portion is defined by an arc or curve in the elongated shaft.

In some implementations, the proximal end of the elongated shaft defines a first shaft section and the distal end of the elongated shaft defines a second shaft section.

In some implementations, the bent portion of the elongated shaft is defined by the connection between the first shaft section and the second shaft section.

In some implementations, an angle of the bent portion of the elongated shaft is fixed.

In some implementations, the angle is 15°.

In some implementations, the connection between the bent portion of the elongated shaft is articulable such that the tip is movable with respect to the elongated shaft and such that an angle of the bent portion of the elongated shaft is variable.

In some implementations, the probe further includes wiring that extends through the elongated shaft and the tip and connects to the one or more electrodes.

In some implementations, the probe further includes at least one sensor at the tip.

In some implementations, the sensor is one of a temperature sensor, an impedance sensor, a pressure sensor, a current sensor, a position sensor, or a movement sensor.

In some implementations, the one or more electrodes include oval or circular contacts.

In some implementations, the one or more electrodes include a first circular contact and a second circular contact, the second circular contact surrounding the first circular contact.

In some implementations, the first circular contact and the second circular contact are separated by an interelectrode distance of 0.1 mm to 10 mm.

In some implementations, the tip is at least partially hollow.

In some implementations, the probe further includes one or more fluid circulation channels that extend internally to and through the elongated shaft to respective outlets at the distal end of the elongate shaft, wherein the one or more fluid circulation channels are configured to circulate a fluid through the tip for transferring heat generated by application of the RF energy by the one or more electrodes.

In some implementations, the elongated shaft is a first shaft and the tip is a first tip, the probe further including: a second elongated shaft that extends from a proximal end to a distal end, the second elongated shaft including a bent portion at the distal end; and a second tip positioned on the distal end of the second elongated shaft after the bent portion, the second tip including an active side and an inactive side, the inactive side opposite the active side and including an insulating material, the active side including one or more second electrodes extending therefrom for delivering RF energy to the target nerve.

In some implementations, the probe further includes a handle positioned on the proximal end of the elongated shaft.

In some implementations, the handle includes orientation markings formed on an exterior surface, wherein the orientation markings correspond to the active side and the inactive side of the tip.

Another implementation of the present disclosure is a kit including: a probe including: an elongated shaft that extends from a proximal end to a distal end, the elongated shaft including a bent portion at the distal end; and a tip positioned on the distal end of the elongated shaft after the bent portion, the tip including an active side and an inactive side, the inactive side opposite the active side and including an insulating material, the active side including one or more electrodes extending therefrom for delivering RF energy to a target nerve; and a cannula through which the elongated shaft and the tip of the probe extend to facilitate insertion of the probe into a patient.

In some implementations, the kit further includes: a second probe including: a second elongated shaft that extends from a proximal end to a distal end, the second elongated shaft including a bent portion at the distal end; and a second tip positioned on the distal end of the second elongated shaft after the bent portion, the second tip including an active side and an inactive side, the inactive side opposite the active side and including an insulating material, the active side including one or more second electrodes extending therefrom for delivering RF energy to the target nerve; wherein the second elongated shaft and the second tip of the second probe further extend through the cannula to facilitate insertion of the second probe into the patient.

### BRIEF DESCRIPTION OF THE DRAWINGS

The accompanying figures, which are incorporated herein and form part of the specification, illustrate implementations of a device and method to treat CLBP via sinuvertebral nerve ablation. Together with the description, the figures further serve to explain the principles of the device and method to treat chronic lower back pain via sinuvertebral nerve ablation described herein and thereby enable a person skilled in the pertinent art to make and use the device and method to treat chronic lower back pain via sinuvertebral nerve ablation.

FIG. 1 is a diagram of the anatomy of vertebral bodies in relation to the sinuvertebral nerve, according to some implementations.

FIG. 2 is a diagram of a probe tip for ablating the sinuvertebral nerve, according to some implementations.

FIGS. 3 and 4 are diagrams illustrating alternate views of the probe tip of FIG. 2, according to some implementations.

FIGS. 5A-5C are diagrams that illustrate the unsheathing (e.g., release from the cannula) of a dual-prong probe, according to some implementations.

FIGS. 6A and 6B are diagrams of a stylet for use with the probe described herein, according to some implementations.

FIGS. 7A and 7B are diagrams that illustrate the use of a cannula to enter the bony foramen and to position a probe tip at the ablative site, according to some implementations.

FIG. 8 is a diagram of a probe head having indwelling channels, according to some implementations.

### DETAILED DESCRIPTION

As mentioned above, lower back pain is a debilitating pathology that has been shown to affect 84% of people at least once in their lifetime. At any given time, 30% of the general population of the United States is actively dealing with lower back pain. In more advanced cases, people seek medical care for their symptoms, resulting in 52 million medical consultations per year, ranging from hospital visits, and physician check-ups, to emergency care. While most patients will experience recovery, 15% of these patients do not, and are deemed to have chronic lower back pain.

The transpedicular approach for basivertberal nerve ablation involves advancing a probe through spinal bone to access the target nerve, and then setting it to a high temperature to ablate it. In whole, this procedure has quite a bit of risk, requiring the provider to drill a hole through a degenerative bone at each level. This process takes 30 minutes and is generally repeated two to three times. Furthermore, Overall, this treatment presents risk to the patient, a high surgical burden for the physician, and fails to comprehensively treat CLBP, as noted by only having a partial decrease in ODI score. Since the basivertebral nerve is only found to innervate the vertebral body and endplate, its ablation only treats the vertebrogenic component of pain.

Referring generally to the figures, a device and methods are shown that can treat chronic lower back pain (CLBP) originating from the vertebral bodies, intervertebral disc, and other nearby structures via a minimally invasive, timely, easily performed, cost-effective approach, and without damaging diseased tissue. In particular, the disclosed device and methods to chronic lower back pain (CLBP) via sinuvertebral nerve ablation. In some implementations, the disclosed device and methods denervate downstream structures, without damaging structures (e.g., nerve roots, ramus, cauda equina, spinal cord) proximal to the bony foramen and/or ablation site and with a simple posterolateral approach that uses ordinary imaging techniques that are routinely practiced by physicians. As described in greater detail below, treatment via the disclosed device and methods can affect multiple levels and structures with a single ablation of the sinuvertebral nerve, leading to a more effective solution for CLBP.

The disclosed device generally includes an ablation probe configured to be inserted through the bony foramen via a cannula. In some implementations, the cannula may have electrical and/or thermal insulating properties. An advantage of cannula insertion is visibility on a fluoroscope and tactile feel to the provider, such as provided by a Tuohy needle or the like. However, it should be understood that the ablation probe described herein can be used with any insertion or treatment method and is not limited solely to insertion via a cannula. Further, any of the disclosed implementations may be used with a separate cannula, with a kit that comprises the cannula and a probe, or as a probe alone. After insertion, the disclosed probe is generally configured to be extended, allowing an end of the probe to “grab” onto the inner sides of the pedicles at the region of the sinuvertebral nerve.

Generally, the disclosed probe includes one or more electrical contacts used to deliver thermally ablative, radiofrequency (RF) electrical energy. The electrodes described herein may be separate or in a single component. The probe may also constitute a single implementation comprising a cannula, stylet, and electrode. Placement of the probe may be done via a posterior lateral approach (e.g., 7-8 cm deep in the average person). A probe according to the principles described herein allows for steering current to apply the current to tissue, which results in a temperature increase and tissue ablation. In some implementations, the disclosed device is provided in a kit that includes a probe, cannula, and stylet, and (optionally) an RF generator. The probe tip is used to ablate the nerve, and the configurations described herein provide steerability such that ablation of portions of the patient's anatomy other than the target nerve is reduced or eliminated.

The device and method are designed to safely ablate the sinuvertebral nerve by denervating the downstream structures, without damaging structures (nerve roots, ramus, cauda equina, spinal cord) proximal to the bony foramen/ablation site, and with a simple posterolateral approach that uses ordinary imaging techniques that are routinely practiced by IVP physicians. The presently described devices and methods may provide certain advantages, including the ability to treat multiple vertebrae in the same procedure and anatomical isolation during treatment, thereby providing treatment that avoids damaging surrounding anatomical structures. The probe described herein may be placed safely behind the nerve, with thermal and electrical isolation making it possible to provide treatment that is less destructive than previous treatments. According to principles described herein, treatment may be made outside of the spinal canal, thus avoiding the spinal nerve and without entering diseased bone. Implementations of the probe described herein allow for treatment with only nerve ablations. For example, implementations of the device described herein may be provided by cannula insertion or other known methods.

### Sinuvertebral Nerve

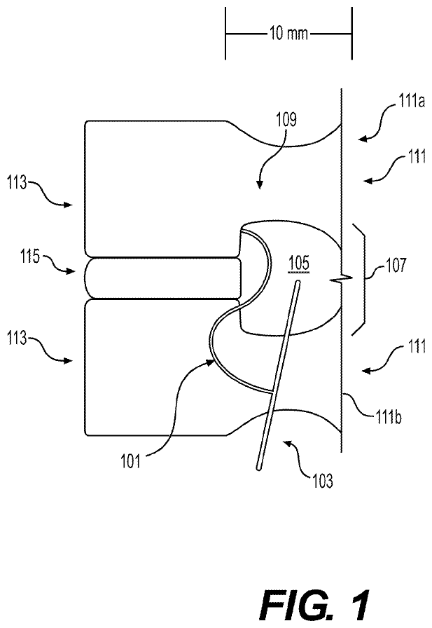

Referring to FIG. 1, a diagram illustrating the sinuvertebral nerve 101 with respect to surrounding biological structures is shown, according to some implementations. The sinuvertebral nerve 101 is a small recurrent branch of the spinal nerve 103. As shown, the sinuvertebral nerve 101 reenters the spinal canal 105 through the intervertebral foramen 107 and passes caudally and anteriorly around a base 109 of a pedicle 111a. After the sinuvertebral nerve 101 exits through the intervertebral foramen 107, it turns back on itself and re-enters the spinal canal 105 passing back through the intervertebral foramen 107. The sinuvertebral nerve 101 travels anteriorly and caudally around the base 109 of a pedicle 111a before splitting to innervate various structures including the dura mater (not shown), vertebral bodies 113, intervertebral discs 115, and the posterior longitudinal ligament (not shown). A single sinuvertebral nerve will innervate structures a few segments caudal and cephalad to the origination level and originates at each neurological segment. Further, the sinuvertebral nerve branches into the basivertebral nerve upon entry into the vertebral body. The sinuvertebral nerve originates centrally from the ventral ramus and can be accessed by a needle-like probe inserted through soft tissue (e.g., posterolateral approach, fluoroscopy).

### Probe Configurations

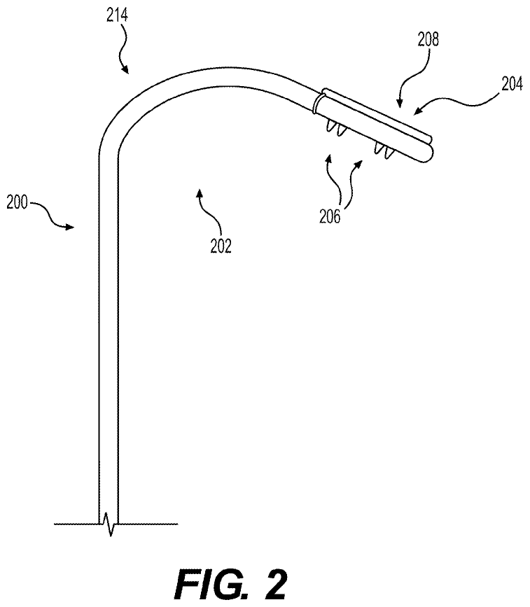

Referring now to FIG. 2, a diagram of a distal end or “head” of a probe 200 is shown, according to some implementations. Generally, probe 200 is configured to apply RF energy to a nerve (e.g., the sinuvertebral nerve 101), e.g., to ablate the nerve. To this point, it should be understood that FIG. 2 only illustrates a portion of probe 200 (e.g., the distal end portion). Probe 200 includes a probe shaft 214 which extends from a proximal end (not shown) to the illustrated distal end or “head.” Generally, probe shaft 214 may be formed of any suitable materials, such as plastic or metal; however, it should be appreciated that probe shaft 214 is generally made of a material that is safe for insertion into the human body. As shown, probe 200 generally has a curved or hooked end—referred to herein as hooked end 202—with an inner side and an outer side (e.g., the inner side of an arc and an outer side of an arc formed by hooked end 202). As shown, hooked end 202 is generally defined by a curved or arced portion of probe shaft 214. Although the term “arc” is used for the purposes of this discussion, the actual shape of the curve or hook at the end of probe 200 is not limited to an arcuate or curved profile.

As shown, the distal end of probe 200 (e.g., after the curved portion of hooked end 202) includes a probe tip 204, which includes an active electrode or multiple electrodes—collectively shown as electrodes 206. Electrodes 206 are positioned on an inner side of hooked end 202, e.g., to contact the sinuvertebral nerve when inserted into a patient. In some implementations, electrodes 206 have smooth edges to provide for a uniform current density. Probe tip 204 may further include one or more inactive regions, e.g., that are not configured as electrodes, including an outer side of probe tip 204 (e.g., opposite the inner side). As such, the inner side (e.g., as viewed in FIG. 2) may be termed an “active side” of probe tip 204 and the outer side may be termed the “inactive side” of probe tip 204. In some implementations, the outer side of probe tip 204 (e.g., opposite from the inner side which includes electrodes 206) includes an insulating material 208 to protect anatomical structures that are not intended to be ablated (e.g., structures in the vicinity of a target nerve). Generally, insulating material 208 can be formed of any suitable insulating material, such as ceramic, polyimide, PTFE coatings, or the like. It should also be appreciated that insulating material 208 can be selected to provide electrical insulation, thermal insulation, or both.

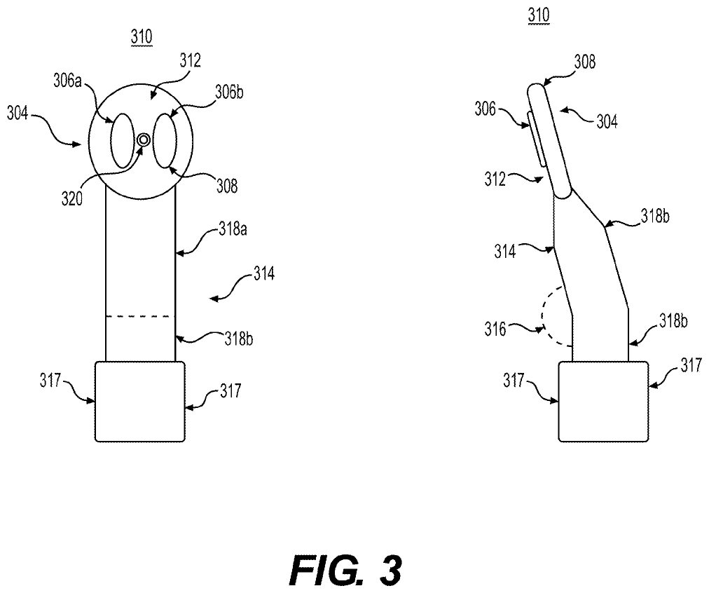

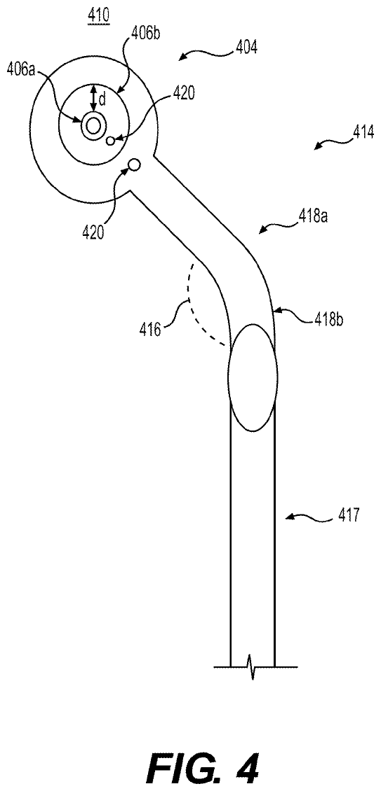

While not illustrated in FIG. 2, but as shown in FIGS. 3 and 4 described below, probe tip 204 can be circular, oval, disk, or spade-shaped when viewed from a front side (e.g., when looking at the active side of probe tip 204). For example, in FIG. 2, probe tip 204 may be shaped as a disk. However, it should be appreciated that probe tip 204 can be configured in other shapes depending on the implementation. It should be appreciated that probe 200 can further include wiring for delivering electrical stimulation and/or transmitting signals/data (e.g., from sensors). The wiring (not shown) may extend internally to and through probe shaft 214 and into probe tip 204, where it is electrically connected to at least electrodes 206. In some implementations, probe tip 204 is hollow and therefore defines an internal cavity through which wiring is routed, e.g., to electrodes 206. In some implementations, all of probe tip 204—with the exception of electrodes 206—is formed of an insulating material (e.g., insulating material 208). Therefore, the wiring may extend through probe shaft 214 and insulating material 208.

Referring now to FIG. 3, another configuration of a probe head 310 according to principles described herein is shown. In some implementations, probe head 310 is an alternative configuration of probe tip 204, as described above. As shown, in some implementations, probe head 310 extends from the end of a cannula 317. In other words, probe head 310 may be passed through cannula 317, e.g., for placement prior to stimulation. However, other implementations are contemplated herein in which probe head 310 is advanced/placed without a cannula. It should also be appreciated that probe head 310 may be a distal part of a larger device, such as a probe that includes an elongated shaft (not shown). To this point, probe head 310 is shown to include probe tip 304 and a probe shaft 314.

Probe tip 304 may be “flattened”, such that at least one face (e.g., face 312) of probe tip 304 is substantially planar. In some implementations, such as the one shown in FIG. 3, probe tip 304 is shaped in a circular or spade. For example, in the illustrated implementation of FIG. 3, probe tip 304 has an oval or circular appearance when viewed from a front of the probe. However, it should be appreciated that probe tip 304 may have any shape depending on the implementation. As shown, face 312 generally includes at least one electrode positioned thereon; however, in some implementations, probe head 310 can include multiple electrodes—shown as electrodes 306a, 306b—positioned on face 312 of probe tip 304. In some implementations, electrodes 306a, 306b are between 1 mm and 10 mm in size; however, the present disclosure is not intended to be limiting in this regard.

In some implementations, probe tip 304 includes one or more inactive regions, e.g., at the distal end of probe head 310 or on a face opposite from face 312. Face 312 of probe tip 304 may further include insulation 308 such that probe tip 304 is thermally and electrically insulated to reduce or prevent heat and electrical current from escaping the space between electrodes 306a, 306b, and ancillary tissues (e.g., spinal nerve root, dura mater, spinal cord, cauda equina, etc.). In some implementations, insulation 308 includes ceramic, polyimide, PTFE coatings, or the like; however, any suitable insulting material is considered herein.

Probe shaft 314 is shown to generally include two portions, including a distal probe head portion 318a and proximal probe head portion 318b. In some implementations, distal probe head portion 318a and proximal probe head portion 318b are configured at an angle 316 with respect to one another. Angle 316 may be formed by a gradual bend in probe shaft 314 or by an angled intersection of ends of the distal probe head portion 318a and proximal probe head portion 318b. As illustrated, distal probe head portion 318a is distal to probe shaft potion 318b and is oriented at the same or similar angle to probe tip 304. For example, probe tip 304 of the probe may have a slight (e.g., less than 15°) with respect to proximal probe head portion 318b. In some implementations, the respective angle of the probe tip with respect to proximal probe head portion 318b is imparted by the angle of distal probe head portion 318a with respect to proximal probe head portion 318b. In some implementations, a vertex of angle 316 may be 5 mm to 15 mm from a tip of electrodes 306a, 306b. In some implementations, a shaft length of probe shaft 314 may be 5-15 cm measured from the probe base to the vertex.

Generally, angle 316 is selected such that probe tip 304 (e.g., containing electrodes 306a, 306b) sits flush against the vertebral bone and overtop of the sinuvertebral nerve fibers (as shown in FIG. 1) when positioned for stimulation, and the backside of probe tip 304 (e.g., the insulated/protected side having an insulated backing formed of insulation 308) is positioned against dura mater and other nervous tissue (not shown). While illustrated here as being 15°, the degree of angle 316 is not so limited and may be set as appropriate to provide flush positioning and appropriate protection of the patient tissue. For example, the illustrated 15° bend allows for a 45° posterior lateral insertion, but another insertion positioning may benefit from a different angle 316. In some implementations, the portions of probe tip head 310 distal of angle 316 are insulated to protect surrounding tissue. In some implementations, the relative angle of probe tip 304 with respect to proximal probe head portion 318b is controllable by hand controls (not shown). For example, probe shaft 314 may be made of flexible material to allow relative motion of distal probe head portion 318a, proximal probe head portion 318b, or both, to move the relative angle from 0° to 90°.

In some implementations, probe tip 304 includes at least one sensor 320. In some such implementations, sensor 320 is a temperature sensor for measuring the temperature of the environment of probe tip 304, e.g., during the application of energy. In other such implementations, sensor 320 is one of a pressure sensor, a current sensor, an impedance sensor, a position and/or movement sensor, and/or the like. It will be appreciated that, in some implementations, sensor 320 includes more than one sensor for measuring multiple parameters and/or probe tip 304 may include additional sensors to measure these parameters. In some implementations, probe head 310 includes indwelling channels to circulate a coolant (e.g., water) or to enable a phase-change reaction (e.g., wax). These indwelling channels are shown in FIG. 8, which is described in greater detail below. Generally, a coolant can serve to better control temperatures surrounding probe tip 304, e.g., to protect heating of ancillary tissues and to maximize energy delivered through the electric contacts.

Referring now to FIG. 4, yet another configuration of a probe head 410 according to principles described herein is shown. In some implementations, probe head 410 is an alternative configuration of probe tip 204 and/or probe head 310, as described above. Similar to probe head 310 as described above, probe head 410 generally includes a probe tip 404 and a probe shaft 414. As shown, in some implementations, probe head 410 extends from the end of a cannula 417. In other words, probe head 410 may be passed through cannula 417, e.g., for placement prior to stimulation. However, other implementations are contemplated herein in which probe head 410 is advanced/placed without a cannula. It should also be appreciated that probe head 410 may be a distal part of a larger device, such as a probe that includes an elongated shaft (not shown).

Probe tip 404, as shown, may have a face or cross-sectional shape of a circle, oval, or spade, although the present disclosure is not intended to be limiting in this regard. In some implementations, probe tip 404 is flattened in shape, e.g., when viewed from the side. Additionally, in some implementations, probe tip 404 is thermally and electrically insulated to prevent heat and electrical current from escaping the space between electrical contacts—shown as contacts 406a, 406b—and ancillary tissues (i.e., spinal nerve root, dura mater, spinal cord, cauda equina, etc.). In some implementations, contacts 406a, 406b are configured as a single circular contact (e.g., contact 406a) that is surrounded by a second circular contact (e.g., contact 406b). In some such implementations, contacts 406a, 406b are generally centered on probe tip 404. In some implementations, contacts 406a, 406b are separated by an interelectrode distance, d, of approximately 0.1-10 mm when configured for the treatment of the sinuvertebral nerve.

Probe shaft 414 is shown to include a distal probe shaft portion 418a and a proximal probe shaft portion 418b. In some implementations, distal probe shaft portion 418a and probe shaft portion 418b are at an angle 416 with respect to one another. Angle 416 may be formed by a gradual bend in probe shaft 414 or by an angled intersection of ends of distal probe shaft portion 418a and probe shaft portion 418b. As shown, probe shaft portion 418a is distal to probe shaft portion 418b and is oriented at the same or similar angle to probe tip 404. Probe head 410 may have a slight (e.g., less than 15°)bend 416 to it, such that the portion containing contacts 406a, 406b (e.g., probe tip 404) sits flush against the vertebral bone and overtop of the sinuvertebral nerve fibers, and the backside of probe tip 404 (e.g., the side opposite from the face that includes contacts 406a, 406b) is positioned against dura mater and other nervous tissue. In some implementations, the back side of probe tip 404 includes insulation (e.g., similar to 308) to protect non-target tissues.

While illustrated here as being 15°, the degree of angle 416 is not so limited and may be selected as appropriate to provide flush positioning and appropriate protection of the patient tissue. For example, the illustrated 15° bend allows for a 45° posterior lateral insertion, but other insertion positioning may benefit from a different angle 416. The portions of probe tip head 410 distal of angle 416 may be insulated to protect surrounding tissue, as mentioned above. In some implementations, the relative angle of distal probe shaft portion 418a with respect to probe shaft portion 418b is controllable by hand controls (not shown). As such, probe shaft 414 may be made of flexible material to allow relative motion of distal probe shaft portion 418a and probe shaft portion 418b to move the relative angle from 0° to 90°.

In some implementations, probe tip 404 includes one or more sensors 420. In some such implementations, sensors 420 are configured to measure temperature(s) between and/or surrounding contacts 406a, 406b. The sensed temperatures may be used to assure thermal safety and as input to drive energy delivery. In other such implementations, sensors 420 are or include pressure sensors, current sensors, impedance sensors, position and/or movement sensors, and/or the like. It will be appreciated that, in some implementations, sensors 420 includes more than one type of sensor for measuring multiple parameters. In some implementations, probe head 410 includes indwelling channels (shown in FIG. 8) to circulate a coolant (e.g., water) or to enable a phase-change reaction (e.g., wax). As mentioned above, a coolant serves to better control temperatures surrounding the probe-tip, to protect heating of ancillary tissues and to maximize energy delivered through the electric contacts.

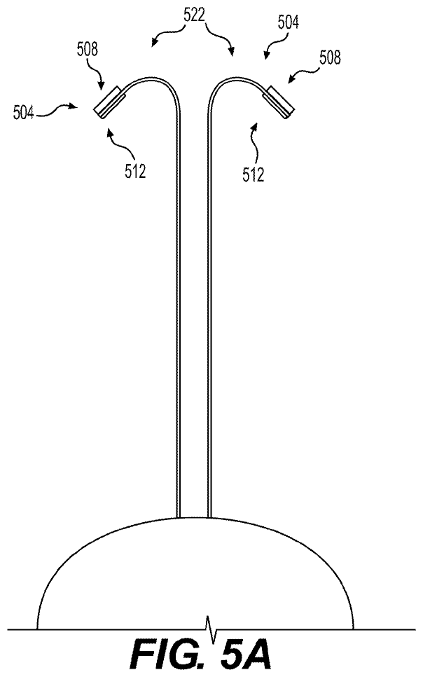

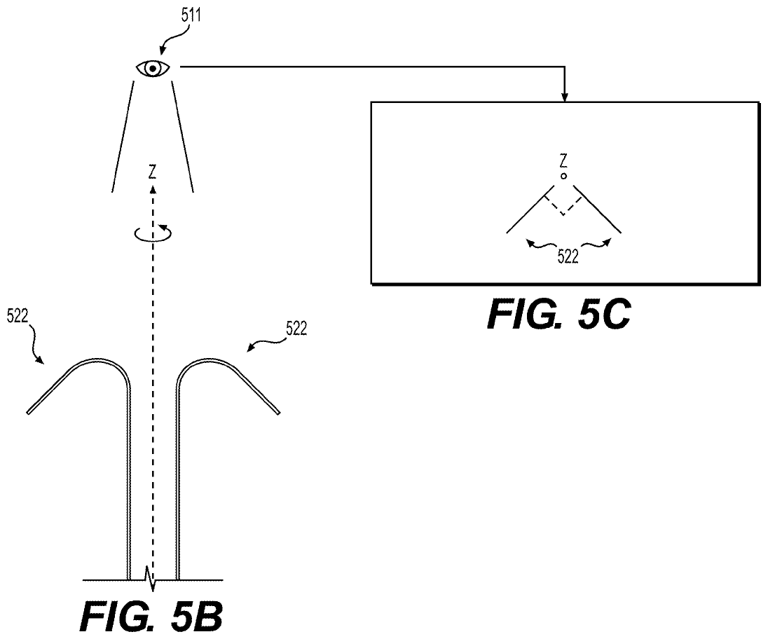

Referring now to FIGS. 5A-5C, diagrams that illustrate the unsheathing (e.g., release from the cannula) of a dual-prong probe are shown, according to various implementations. Generally, in any implementation of the device described herein, the electrical contacts may be configured as movable tines 522 (e.g., rather than the electrodes shown in FIGS. 2-4) that are deployed on one side of a probe tip—shown as probe tips 504—and that are advanced into an ablation zone. In some implementations, probe tips 504 have an insulated side 508 and an active side 512, e.g., similar to the probe heads described above with respect to FIGS. 2-4. In some such implementations, movable tines 522 are covered (e.g., before being deployed/extended) by an insulated backing (e.g., insulation). Additionally, movable tines 522 may be connected via an insulated sheath.

As shown, the movable tines 522 may be configured as two “fishing hook” shaped connectors, e.g., as in FIG. 2. In such implementations, the probe, after entering the intervertebral foramen will hook around the back side and make a connection to the bone, e.g., as illustrated in FIG. 1. As shown, when unsheathing or extending (e.g., releasing from a cannula), movable tines 522 move orthogonal to one another, e.g., in relation to the principle axis of the sheathing. Looking to FIG. 5B, for example, the major axis Z is shown as reference. It can be seen that the probe is illustrated as running substantially or generally parallel to the major axis Z before hooking away from the axis Z. The inset picture, FIG. 5C, shows the angle at which movable tines 522 extend from the Z axis in relation to the right angle marked.

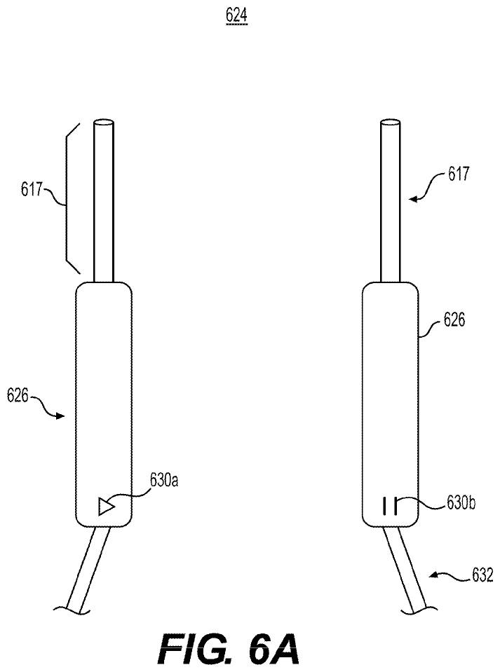

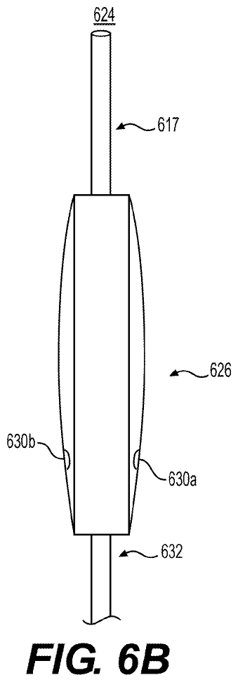

Referring now to FIGS. 6A and 6B, a handle device 624—also referred to herein as a “stylet”—for use with the probe(s) described herein is shown, according to some implementations. Generally, handle device 624 may include a held portion 626 from which a cannula 617 extends. As shown, cannula 617 may be connected to held portion 626 or, in some implementations, cannula 617 may be separate from held portion 626. Together, cannula 617 and held portion 626 facilitate the insertion of any of probe(s) described herein. In some implementations, probe 200 (e.g., with any of the probe tip configurations described herein) can extend through cannula 617 and held portion 626 during insertion. In other implementations, probe 200 extends from held portion 626 and through cannula 617.

Held portion 626 may have a front button 630a and back button 630b on it so that the orientation of the probe tip (not shown) within cannula 617 will be easily identifiable to a user (e.g., a physician). In particular, front button 630a and back button 630b may be different markings such that a user can easily identify the active and inactive sides of probe 200. In the example shown, front button 630a may indicate the active side of probe 200 (e.g., containing electrodes 206) and back button 630b may indicate the inactive side of probe 200 (e.g., insulating material 208).

An electrical connection for powering the electrodes of the probe (not shown) may be provided by a cable 632 that extends into and/or through handle device 624 and/or an attached probe shaft (not shown). In some implementations, cable 632 terminated internally to handle device 624; thus, handle device 624 and/or the probe shaft may include appropriate internal wiring within the handle 624. In other implementations, an electrical power supply may be provided within handle device 624 or held portion 626. Although not shown, held portion 626 may have an ergonomic shape, allowing for ease of handling and facilitating placement of the probe. For example, held portion 626 may have rounded front and back sides for comfort of holding. Flattened edges on the sides may further assist proper orientation of active and insulating sides of the probe tip. Buttons 630a, 630b may have a raised profile to be identifiable by touch. The profiles of buttons 630a, 630b may be different to allow for knowing which side of the handle is which by touch.

While also not illustrated, it should be appreciated that the probe(s) described herein (e.g., shown in FIGS. 2-5) may be connected to an external radiofrequency (RF) generator prior to use. The external RF generator is generally configured to generate and supply controlled RF energy to perform ablation. Specifically, the external RF generator may supply RF energy to the electrodes or electrical contacts of the probe (e.g., 206, 306a, 306b, 406a, 406b). The external RF generator may produce RF energy at an acceptable frequency to perform ablation, for example, but not limited to, approximately 480 kHz. However, the external RF generator may more generally produce RF energy in a range of 100 kHz to 1 MHz. In some implementations, if the cannula and/or stylet of a probe are equipped with electrical channels, they will also be connected to the external RF generator. In some implementations, the probe, cannula, and/or stylet are connected to the external RF generator via a common cord (e.g., cable 632). To this point, it should also be appreciated that the probe(s) described herein may be configured in a kit that includes at least one of a cannula, a stylet, or an external RF generator. For example, probe 200 may be part of a kit that includes a cannula to facilitate insertion into a patient. As discussed above, the cannula may include one or more open channels to pass a stylet, fluid, visual scope, or other implements.

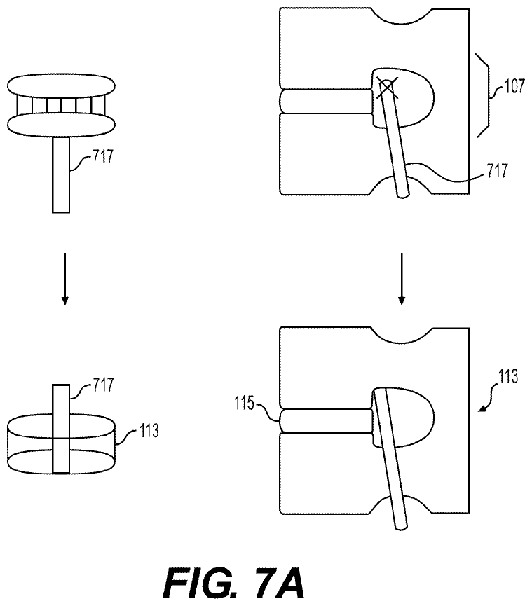

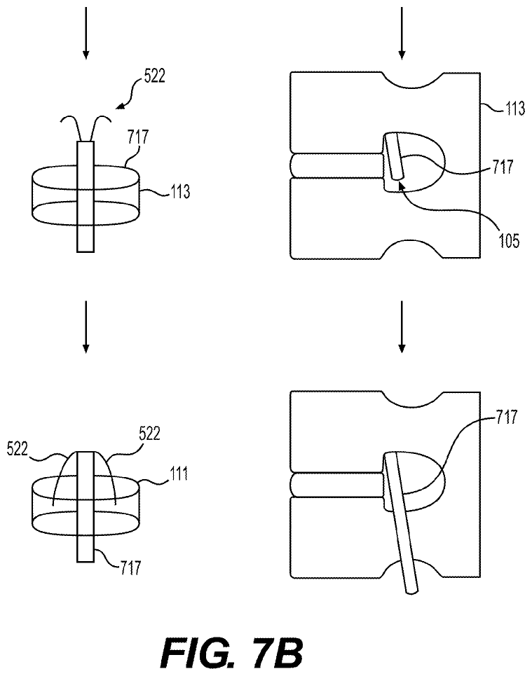

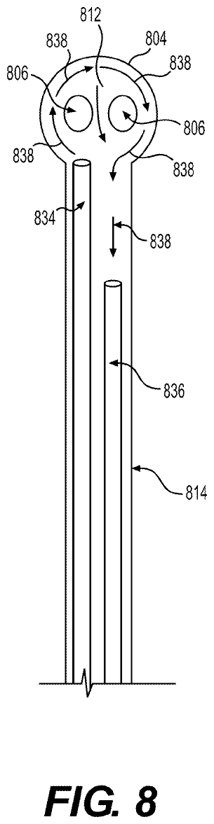

Referring now to FIGS. 7A and 7B, an example cannula-assisted insertion process for the probe described herein is shown, according to some implementations. Generally, a cannula 717 may be used to enter the bony foramen 107 and to position the probe tip at the ablative site (FIGS. 7A and 7B). FIG. 7A, specifically, illustrates cannula 717 approaching the intervertebral foramen. At this stage, the probe is retained/contained within cannula 717. The point at which the probe is expected to pass through the foramen is denoted in FIG. 7A by the “X” in the right side of the figure and corresponds to an area at which the sinuvertebral nerve reenters the foramen. The cannulated probe is advanced through the opening and checked to see if it is in the correct position. This double-checking provides verification that the probe contacts are oriented appropriately for proper contact after unsheathing and/or during ablation. Double-checking may be assisted through the use of markings 630a and 630b on the handle (FIGS. 6A and 6B).

In the illustrated implementation of FIGS. 7A and 7B, movable tines 522 are unsheathed and the active “fish hook” probe tips are revealed from the tip of the cannula 717. The probe can then be pulled back to make contact with bone at the treatment target area. The hooks wrap around the base of the pedicle 111 at the caudal and anterior portion of the foramen. By connecting energy, the sinuvertebral nerve can be ablated against the inside bone of the intervertebral foramen 107, while insulating the remainder of the spinal canal 105 from the energy.

If used, cannula 717 may have one or more open channels designed to pass a stylet, fluid, visual scope, or other tools (e.g., microdissection tools). Cannula 717 and stylet may be equipped with electrical channels extending to their tips, capable of delivering electrical stimulation through them and determining tissue impedance (FIG. 7A). The use of the electrical channels will help guide the cannula and stylet to the foramen without entering a nerve, and to identify nearby tissue types (fat; connective tissue; scar, etc.). In some implementations, imaging (e.g., fluoroscopy, ultrasound, hyperechoic, or any other imaging technique that allows the probe to be visible to a user) may be used to assist in orienting the probe within the body. In addition, some type of stimulation to the nerve may be provided by the probe tip to verify placement. For example, a sensory response and/or muscle twitch caused by a brief electrical pulse of the probe at low energy levels could identify whether the probe is electrically proximate to the target nerve.

Referring now to FIG. 8, an example probe head 804 that includes indwelling channels is shown, according to some implementations. As mentioned above, indwelling channels can be included in any of the probe configurations described herein; however, only one specific configuration is illustrated for brevity. It should therefore be appreciated that the following description is not intended to be limiting. The indwelling channels are illustrated in FIG. 8 as a coolant input tube 834 and a coolant return tube 836 that circulate a coolant (e.g., water) or to enable a phase-change reaction (e.g., wax). Coolant input tube 834 and coolant return tube 836 are shown to extend toward probe tip 812 via a probe shaft 814. Coolant flows from coolant input tube 834 and is deposited to an interior space of probe tip 812 (e.g., a cavity within probe tip 812), thereby circulating behind electrodes 806. In this manner, the heat generated by the application of RF by electrodes 806 is transferred to the coolant. The heated coolant then flows from the cavity at probe tip 812 into coolant return tube 836, where it is returned to a pump, reservoir, or other component. Example coolant flow 838 is indicated by arrows in FIG. 8. The coolant will serve to better control temperatures surrounding the probe tip 812, to protect heating of ancillary tissues and to maximize energy delivery through the electric contacts.

### Additional Implementations

As generally described herein, the disclosed system and devices can be used for the treatment of CLBP. Alternatively, the disclosed system and devices can be used for the treatment of cervicogenic headaches and other ailments. Cervicogenic headaches are triggered by sinuvertebral nerve compression and irritation in the C1-C3 vertebral level. These headaches are also implicated with potentially being a cause of migraines. This treatment paradigm, normally used for CLBP in the lumbar region, is easily placeable in the cervical level, allowing for preventative treatment of cervicogenic headaches and cervicogenic headache-caused migraines. This alternative method would take the same approach and use the same probe and stimulator but would require an entry at the cervical level and different waveform parameters.

Treatment using the probe of any of the above-described implementations may include some or all of the following steps:
I. Cannula and stylet are transcutaneously placed via fluoroscopic guidance to the caudal border of the bony foramen, and caudal again, to the spinal nerve root, at the targeted level.
II. Electrical stimulation and impedance checks may be performed through the cannula and stylet as necessary to identify their location and the nearby tissue types.
III Microdissection tools or hydro-dissection techniques may be delivered through the cannula and used to further open the foramen to assure safe passage of the cannula and probe.
IV. Chemical nerve blocks or insulating fluids may be used prior to or after the therapy, delivered through the cannula.
V. The stylet is removed from the cannula and replaced with a probe.
VI. The probe's tip is positioned near the target nerve via fluoroscopic and electrical guidance. The electrical contacts may contact the bone, and the insulated regions of the probe will be interposed between the electrical contacts and non-targeted tissues.
VII. Traditional electrical stimulation will be delivered to determine target proximity; impedance values stored and used similarly.
VIII. RF energy is delivered to the target nerve via the probe.
IX. After running the desired RF treatment, the transcutaneously-placed leads will be removed.
X. Desirably, the generator and leads will be reused.

It should be appreciated that the disclosed system and devices (e.g., probes) can be used in any of the following treatment regions:
I. Intervertebral foramen anterior and caudal region.
II. Targeting the sinuvertebral nerve branch that is recurrent through this point.
III. SVN has downstream branches such as BVN, IVD, vertebral bodies, vertebral endplates, dura mater, facet joints.
IV. All of these downstream targets may feel the effect of this ablation.
   However, it should be noted that this list is not intended to be limiting.

As mentioned above, the sinuvertebral nerve can be ablated as it re-enters the intervertebral foramen and before it begins to branch. A single multi-level CLBP relief is possible with just a single, minimally invasive ablation of the sinuvertebral nerve. By treating upstream of multiple targets associated with CLBP, it is suggested that ablation will provide the patient with more comprehensive pain relief. Since the nerve is located at this foramen, providers will not have to take a risky transdiscal or transpedicular approach to reach it. Finally, since this nerve has ascending and descending branches coming off it after the foramen target point, it is suggested that one ablation can provide multi-level treatment, thus cutting down on the procedural time and surgical burden. Overall, ablation of the sinuvertebral nerve at the intervertebral foramen is a highly promising target that could offer better results than all other treatments, while being much less risky, burdensome, and time intensive as well.

### Configuration of Certain Implementations

The construction and arrangement of the systems and methods as shown in the various exemplary implementations are illustrative only. Although only a few implementations have been described in detail in this disclosure, many modifications are possible (e.g., variations in sizes, dimensions, structures, shapes and proportions of the various elements, values of parameters, mounting arrangements, use of materials, colors, orientations, etc.). For example, the position of elements may be reversed or otherwise varied, and the nature or number of discrete elements or positions may be altered or varied. Accordingly, all such modifications are intended to be included within the scope of the present disclosure. The order or sequence of any process or method steps may be varied or re-sequenced according to alternative implementations. Other substitutions, modifications, changes, and omissions may be made in the design, operating conditions, and arrangement of the exemplary implementations without departing from the scope of the present disclosure.

Although the figures show a specific order of method steps, the order of the steps may differ from what is depicted. Also, two or more steps may be performed concurrently or with partial concurrence. Such variation will depend on the software and hardware systems chosen and on designer choice. All such variations are within the scope of the disclosure. Likewise, software implementations could be accomplished with standard programming techniques with rule-based logic and other logic to accomplish the various connection steps, processing steps, comparison steps and decision steps.

It is to be understood that the methods and systems are not limited to specific synthetic methods, specific components, or to particular compositions. It is also to be understood that the terminology used herein is for the purpose of describing particular implementations only and is not intended to be limiting.

As used in the specification and the appended claims, the singular forms “a,” “an” and “the” include plural referents unless the context clearly dictates otherwise. Ranges may be expressed herein as from “about” one particular value, and/or to “about” another particular value. When such a range is expressed, another implementation includes from the one particular value and/or to the other particular value. Similarly, when values are expressed as approximations, by use of the antecedent “about,” it will be understood that the particular value forms another implementation. It will be further understood that the endpoints of each of the ranges are significant both in relation to the other endpoint, and independently of the other endpoint.

“Optional” or “optionally” means that the subsequently described event or circumstance may or may not occur and that the description includes instances where said event or circumstance occurs and instances where it does not.

Throughout the description and claims of this specification, the word “comprise” and variations of the word, such as “comprising” and “comprises,” means “including but not limited to,” and is not intended to exclude, for example, other additives, components, integers or steps. “Exemplary” means “an example of” and is not intended to convey an indication of a preferred or ideal implementation. “Such as” is not used in a restrictive sense, but for explanatory purposes.

Disclosed are components that can be used to perform the disclosed methods and systems. These and other components are disclosed herein, and it is understood that when combinations, subsets, interactions, groups, etc. of these components are disclosed that while specific reference of each various individual and collective combinations and permutation of these may not be explicitly disclosed, each is specifically contemplated and described herein, for all methods and systems. This applies to all aspects of this application including, but not limited to, steps in disclosed methods. Thus, if there are a variety of additional steps that can be performed it is understood that each of these additional steps can be performed with any specific implementation or combination of implementations of the disclosed methods.

What is claimed is:

1. A probe for radiofrequency (RF) tissue ablation, the probe comprising:
an elongated shaft that extends from a proximal end to a distal end, the elongated shaft comprising a bent portion at the distal end; and
a tip positioned on the distal end of the elongated shaft after the bent portion, the tip comprising an active side and an inactive side, the inactive side opposite the active side and comprising an insulating material, the active side comprising one or more electrodes extending therefrom for delivering RF energy to a target nerve.

2. The probe of claim 1, wherein the bent portion is defined by an arc or curve in the elongated shaft.

3. The probe of claim 1, wherein the proximal end of the elongated shaft defines a first shaft section and the distal end of the elongated shaft defines a second shaft section.

4. The probe of claim 3, wherein the bent portion of the elongated shaft is defined by the connection between the first shaft section and the second shaft section.

5. The probe of claim 4, wherein an angle of the bent portion of the elongated shaft is fixed.

6. The probe of claim 5, wherein the angle is 15°.

7. The probe of claim 4, wherein the connection between the bent portion of the elongated shaft is articulable such that the tip is movable with respect to the elongated shaft and such that an angle of the bent portion of the elongated shaft is variable.

8. The probe of claim 1, further comprising wiring that extends through the elongated shaft and the tip and connects to the one or more electrodes.

9. The probe of claim 1, further comprising at least one sensor at the tip.

10. The probe of claim 9, wherein the sensor is one of a temperature sensor, an impedance sensor, a pressure sensor, a current sensor, a position sensor, or a movement sensor.

11. The probe of claim 1, wherein the one or more electrodes comprise oval or circular contacts.

12. The probe of claim 1, wherein the one or more electrodes comprise a first circular contact and a second circular contact, the second circular contact surrounding the first circular contact.

13. The probe of claim 13, wherein the first circular contact and the second circular contact are separated by an interelectrode distance of 0.1 mm to 10 mm.

14. The probe of claim 1, wherein the tip is at least partially hollow.

15. The probe of claim 14, further comprising one or more fluid circulation channels that extend internally to and through the elongated shaft to respective outlets at the distal end of the elongate shaft, wherein the one or more fluid circulation channels are configured to circulate a fluid through the tip for transferring heat generated by application of the RF energy by the one or more electrodes.

16. The probe of claim 1, wherein the elongated shaft is a first shaft and the tip is a first tip, the probe further comprising:
a second elongated shaft that extends from a proximal end to a distal end, the second elongated shaft comprising a bent portion at the distal end: and
a second tip positioned on the distal end of the second elongated shaft after the bent portion, the second tip comprising an active side and an inactive side, the inactive side opposite the active side and comprising an insulating material, the active side comprising one or more second electrodes extending therefrom for delivering RF energy to the target nerve.

17. The probe of claim 1, further comprising a handle positioned on the proximal end of the elongated shaft.

18. The probe of claim 17, wherein the handle comprises orientation markings formed on an exterior surface, wherein the orientation markings correspond to the active side and the inactive side of the tip.

19. A kit comprising:
a probe comprising:
an elongated shaft that extends from a proximal end to a distal end, the elongated shaft comprising a bent portion at the distal end; and
a tip positioned on the distal end of the elongated shaft after the bent portion, the tip comprising an active side and an inactive side, the inactive side opposite the active side and comprising an insulating material, the active side comprising one or more electrodes extending therefrom for delivering RF energy to a target nerve; and
a cannula through which the elongated shaft and the tip of the probe extend to facilitate insertion of the probe into a patient.

20. The kit of claim 19, wherein the probe is a first probe, the kit further comprising:
a second probe comprising:
a second elongated shaft that extends from a proximal end to a distal end, the second elongated shaft comprising a bent portion at the distal end; and
a second tip positioned on the distal end of the second elongated shaft after the bent portion, the second tip comprising an active side and an inactive side, the inactive side opposite the active side and comprising an insulating material, the active side comprising one or more second electrodes extending therefrom for delivering RF energy to the target nerve:
wherein the second elongated shaft and the second tip of the second probe further extend through the cannula to facilitate insertion of the second probe into the patient.
